(19) United States
(12) Patent Application Publication
(10) Pub. No.: US 2007/0043416 A1
(43) Pub. Date: Feb. 22, 2007

(54) Implantable electrode array

(75) Inventors: Peter Callas, Redwood City, CA (US); Dan Beckman, Indianapolis, IN (US)

Correspondence Address: SCHWEGMAN, LUNDBERG, WOESSNER & KLUTH, P.A., P.O. BOX 2938, MINNEAPOLIS, MN, 55402, US

(73) Assignee: Cardiac Pacemakers, Inc.

(21) Appl. No.: 11/207,420

(22) Filed: Aug. 19, 2005

(51) Int. Cl. A61N 1/362; A61N 1/05

(52) U.S. Cl. 607/129; 607/4

(57) ABSTRACT

An implantable electrode array for providing a plurality of electrodes in communication with a target is described. A flexible, elastic support holds the electrodes in contact with the target tissue. A large number of electrodes can be provided and selectively activated/deactivated for specific functions. These functions include sensing activity of the target tissue and electrical therapy delivery. One technique to control the electrodes is conductive lines or filaments in the support with individual addressing of each electrode to activate/deactivate an individual electrode for a specific function.

### CROSS-REFERENCE TO RELATED APPLICATIONS

This application is related to co-pending, commonly assigned U.S. patent application Ser. No. 11/017,627, titled “EPICARDIAL PATCH INCLUDING ISOLATED EXTRACELLULAR MATRIX WITH PACING ELECTRODES,” filed on Dec. 20, 2004 (Attorney Docket No. 00279.759US1), which is hereby incorporated by reference in their entirety.

### TECHNICAL FIELD

Implanted medical devices that incorporate electrode arrays, for example, implanted cardiac rhythm management devices or systems that include an electrode arrays for sensing and/or treatment.

### BACKGROUND

Implantable medical devices, such as cardiac rhythm management devices, commonly include implanted pacemakers and defibrillator units. These devices include sensing, signal processing and control circuitry, together with a power supply protectively housed in a hermetically sealed case in combination with one or two conductive electrical leads with electrodes designed to connect to the patient's heart muscle tissue. To maintain the integrity of the components in the sealed case, provision must be made for sealed passage of electrical conductors to the exterior for connection to the leads and ultimately to the tissue of interest. This has been typically accomplished by using connector blocks and associated feedthrough conductors located external to the implanted case which, themselves, are typically placed within a sealed lead connector structure of medical grade polymer material. Extensive mapping and sample placements of leads and electrodes may be required to provide the desired therapeutic effect to the patient. Such mapping and sample placements are time consuming, which can lead to further problems for a patient. Moreover, the placement of leads and electrodes is an invasive procedure with all of the possible side effects and dangers of surgery.

### SUMMARY

Several options are possible for the medical device, including, but not limited to, the summary as follows. An implantable electrode array includes a support and a plurality of electrodes on the support. Select one or ones of the electrodes are activated for a particular function. The function is diagnosis in an embodiment. The support is flexible to conform the array to the target site and retain the electrodes at the site. In an embodiment, the support is elastic to conform to the shape of the target site. In one application, the support is adapted to conform to the shape of the heart and hold the electrodes in contact with the epicardium. The support is formed from a plurality of filaments. Some or all of the filaments are electrically conductive to transmit signals through the support to the electrodes, which electrically communicate the adjacent target site. The filaments include a first group of filaments extending in a first direction and a second group of filaments extending in a second direction, the first group crossing the second group at intersections to form a mesh. The electrodes are fixed to the intersections such that each electrode is individually addressable by the crossed first group filament and the second group filament.

In an embodiment, the electrode array cooperates with a control circuit that individually addresses an electrode. The control circuit communicates with electrodes to activate, deactivate, receive sensed signals, or deliver therapeutic signals to select electrodes as the present invention does not require all electrodes to be active at any time. The control circuit may include logic circuits on the electrodes themselves to activate the electrode based on signals received at the electrode. In one embodiment, the logic is an AND gate. The control circuit may include a memory and a processor in communication with the memory.

The electrode array support is generally shaped to conform to the target site. In the application where the site is a heart, then the support is formed in a sack or band shape to generally conform to the outer contours of the heart. Other shapes for the support can be used in for a particular application. In addition to the filaments that may provide electrical communication to the electrodes, the support may include further structures such as a web fixed to the filaments. In an embodiment, the web includes a plurality of generally parallel strands. In an embodiment, the web includes a plurality of interwoven strands. In an embodiment, the web includes a sheet having a plurality of apertures intermediate the filaments and electrodes. In an embodiment, the web is a solid, continuous sheet.

The plurality of electrodes in the electrode array for an embodiment number 2^{N}, where N is a whole number greater than 4. The number of electrodes can be selected from a group of 32, 64, 128, 256, 512, etc., which results in easier addressing of the electrodes based on digital control circuits and addressing. As the number of electrodes increases the size of the electrodes will be smaller and the density will increase. In an embodiment, the electrodes are spaced less than about one millimeter or less from each other.

An implantable medical system includes an electrode array as described herein in combination with a signal generator and an addressing circuit operably connected to the signal generator. The addressing circuit provides signals to selectively activate selected ones of the electrodes. The signal generator provides muscle stimulation signals transmitted though the addressing circuit and electrical conductors of the support to the active electrodes. The signal generator may receive sensed signals from the electrodes through the electrical conductors of the support and the addressing circuit. The signal generator is adapted to produce output signals, such as cardiac rhythm management signals, at the active ones of the plurality of electrodes based on the sensed signals. Accordingly, the system is an active system. In an embodiment, the addressing circuit includes a multiplexer.

The implanted structures as described herein can include a releasable therapeutic agent. The therapeutic agent can include, but is not limited to, at least one selected from the group of anti-arrhythmic drugs, thrombolytic agents, anti-inflammatory, anti-fibrotic agents, antibiotics, and steroids.

In an embodiment an implantable canister is provided to house circuitry safely with a body. The canister may be remote from the target site or adjacent the target site. In an embodiment, the canister is fixed to the electrode array. The canister further houses a power supply such as a battery in an embodiment. The canister may be adapted to receive power from an external source.

These and other embodiments, aspects, advantages, and features will be set forth in part in the description which follows, and in part will become apparent to those skilled in the art by reference to the following description and referenced drawings or by practice thereof. The aspects, advantages, and features are realized and attained by means of the instrumentalities, procedures, and combinations particularly pointed out in the appended claims and their equivalents.

### BRIEF DESCRIPTION OF THE DRAWINGS

In the drawing figures wherein like reference characters depict like parts throughout the same:

FIG. 1 shows a system in accordance with at least one embodiment;

FIG. 2 shows a medical system implanted on a heart in accordance with at least one embodiment;

FIG. 3 shows a view of an embodiment of an electrode array;

FIG. 4 shows a view of an embodiment of an electrode array;

FIG. 5 shows a view of an embodiment of an electrode array;

FIG. 6 shows an embodiment of an electrode;

FIG. 7 shows an enlarge view of an embodiment of the electrode array support.

FIG. 8 shows an enlarge view of an embodiment of the electrode array support.

FIG. 9 shows an enlarge view of an embodiment of the electrode array support.

FIG. 10 shows an enlarged, partial view of an embodiment of the support.

FIG. 11 shows an enlarged, partial view of an embodiment of the support.

FIG. 12 shows an enlarged, partial view of an embodiment of the support.

FIG. 13 shows an enlarged, partial view of an embodiment of the support.

### DETAILED DESCRIPTION

In the following detailed description, reference is made to the accompanying drawings, which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the spirit and scope of the present invention. Therefore, the following detailed description is not to be taken in a limiting sense, and the scope is defined by the appended claims.

It should be noted that references to “an”, “one”, or “various” embodiments in this disclosure are not necessarily to the same embodiment, and such references contemplate more than one embodiment.

The present description may use the terms “above”, “below”, “top” and “bottom” when referring to disclosed embodiments. The present description further may use the terms “upwardly”, “downwardly”, “horizontally”, and “vertically.” These terms refer to directions relative to the substrate and in some instances refer to the surface of the described feature as shown in the drawings. Such terminology will include the words specifically mentioned, derivatives thereof, and words of similar import.

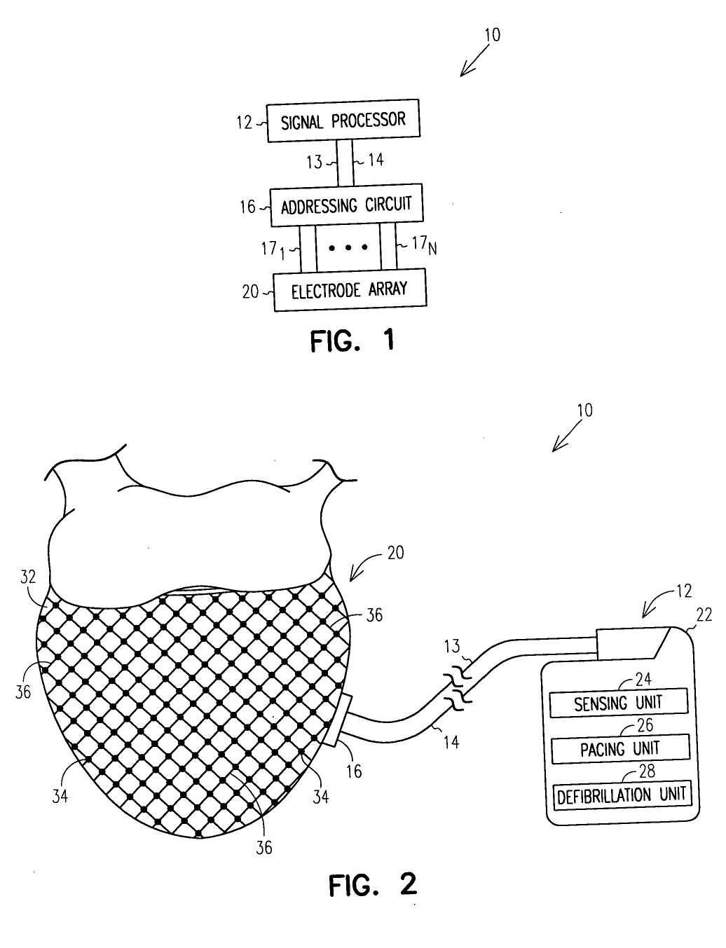

FIG. 1 illustrates an embodiment of a system 10 that includes an electrical signal processing device 12 in electrical communication through signal conductors 13, 14 with an addressing circuit 16. In an output or therapy mode, the addressing circuit 16 interprets signals on conductors 13, 14 and provides signals to a plurality of electric signal lines 17₁, . . . 17_{N} that are connected to an electrode array 20. Addressed electrodes in the electrode array 20 transmit electrical signals to associated therapy sites. In a sensing mode, the electrode array 20 senses environmental conditions at a plurality of sites, each associated with one electrode of the electrode array 20, and through lines 17₁, . . . 17_{N} communicates with addressing circuit 16. Addressing circuit 16 interprets these signals and through conductors 13, 14 transmits signals back to the device 12. System 10 includes, in an embodiment, a programmer or other external system that provides wireless communication signals to communicate with device 12, such as by using radio frequency (RF) or other telemetry signals. The electrode array 20 has a plurality of electrodes that provide rapid mapping and rapid, efficient therapeutic delivery to a patient. Moreover, such an electrode array provide a physician with more pacing options based on a greater number of electrodes and greater control, i.e., activation/deactivation, of electrodes.

FIG. 2 illustrates an embodiment of a medical system 10 having, for instance, an implantable medical device 12, that allows for signals to be sent and/or received from tissue. Tissue includes, for example, a muscle. In an embodiment, the muscle is the heart and the device 12 is a cardiac rhythm management (“CRM”) device. A CRM device includes, without limitation, a pacer, a defibrillator, a cardiac resynchronization therapy (CRT) device, sensing circuitry, diagnostic circuitry, memory, or a combination of such devices. Device 12 may be referred to as an automatic implantable cardioverter defibrillator (AICD). Electrical signal lines 13, 14 connect medical device 12 to addressing circuit 16. The electrical signal lines 13, 14 are also implantable and are sometimes referred to as leads. Addressing circuit 16 is also implantable. The term “implantable” means that the respective component is adapted to be received in a body. A plurality of electric signal lines 17₁, . . . 17_{N} connect addressing circuit 16 to implantable electrode array 20. The electrode array 20 provides therapy to a plurality of selectable locations of a muscle, such as the heart. The electrodes typically deliver cardioversion, defibrillation, pacing, resynchronization therapy, diagnostic sensing, or combinations thereof to at least one chamber of the heart.

Implantable CRM device 12 contains electronics to sense various electrical, acoustical, and/or mechanical signals of a muscle, such as the heart, and also produce current pulses for delivery to the muscle. The pulse sensor and generator also contain electronics and software necessary to detect certain types of heart arrhythmias and to correct for them. In an embodiment, CRM device 12 includes a canister 22 in which houses a sensing unit 24, a pacing unit 26, and a defibrillation unit 28. Canister 22 is a hermetically-sealed container for components of the CRM device 12. Additional electrodes may be located on the can, or on an insulating header, such as for providing unipolar pacing, bi-polar pacing, and/or defibrillation energy, for example, in conjunction with the electrode array 20 disposed epicardially around heart. Electrode array 20, in a sensing mode of the system 10, senses intrinsic electrical activity of a heart. Sensing unit 24, in an embodiment, includes heart sound sensors that convert the detected sounds of the heart into an electrical signal representative of the heart sounds. Typically, an acoustic sensor for a CRM device 12 includes an accelerometer mounted within the can 22. In another sensor example, a microphone is located within the can. In another example, the sensor includes a strain gauge. Sensing unit 24, in an embodiment, includes circuits to detect the electrical activity of the heart. The electrical activity includes the QRS waveform of the heart. Accordingly, sensing unit 24 may receive various inputs. Sensing unit 24 may include signal processing circuits and a signal analyzer circuit that produce signals representative of at least one heart sound or heart electrical signals and performs functions based on the representative signal. Such functions may be implemented by hardware, software, firmware or any combination of hardware, software or firmware.

Pacing unit 26 delivers pacing pulses to protect the heart from injuries associated with ischemic events, including myocardial infarction and/or or to electrically stimulate contraction (pacing) of the heart. According to a cardiac protection pacing algorithm, electrical pacing pulses from pacing unit 26 are delivered to the heart to cause mechanical asynchrony in the myocardial contractions. Pacing unit 26 includes a pulse generator that is connected to the electrode array 20 as described herein to deliver pacing pulses. Moreover, the pacing signals generated by the pacing unit 26 further include addressing signals that activate select electrodes of the electrode array 20 to specifically target the desired locations of the heart that require pacing to improve performance of the heart.

Defibrillation unit 28 delivers electrical signals to reverse certain life threatening arrhythmias, i.e., defribillate or cardiovert. According to a cardiac protection algorithm which receives sensed signals from the sensing unit 24, when a life threatening event is sensed, then the defibrillation unit 28 delivers an electrical shock to the heart. Defibrillation unit 28 includes a pulse generator that is connected to the electrode array 20 as described herein to deliver defibrillation pulses. Like the pacing unit 26, the defibrillation signals further include addressing signals that activate select electrodes of the electrode array 20 to specifically target the desired locations of the heart that require defibrillation to improve performance of the heart. The locations for defibrillation signal delivery may be different than the locations for pacing signal delivery.

Addressing circuit 16 in the embodiment shown in FIG. 2 is fixed to the electrode array 20. Addressing circuit 16 sends/receives signals to/from the CRM device 12. In an embodiment, the addressing circuit 16 includes a multiplexer. In the sensing mode, the multiplexer will receive signals from active electrodes of the electrode array 20 and combine these signals into signals that are transmitted along at least one of the conductors 13, 14. In an embodiment, the transmitted signals are serial. In a therapy mode, the CRM device 12 sends a therapy signal to the multiplexer over conductors 13, 14. The multiplexer then demultiplexes the signal to address selected electrodes and to provide a therapy signal to the addressed electrodes in electrode array 20. In an embodiment, addressing circuit 16 includes a row decoder and a column decoder that are each electrically connected to the respective row and column of the electrode array 20. When at least one row and at least one column are activated, then where these activated rows and columns cross the electrode at the crossing point of the row and the column are activated.

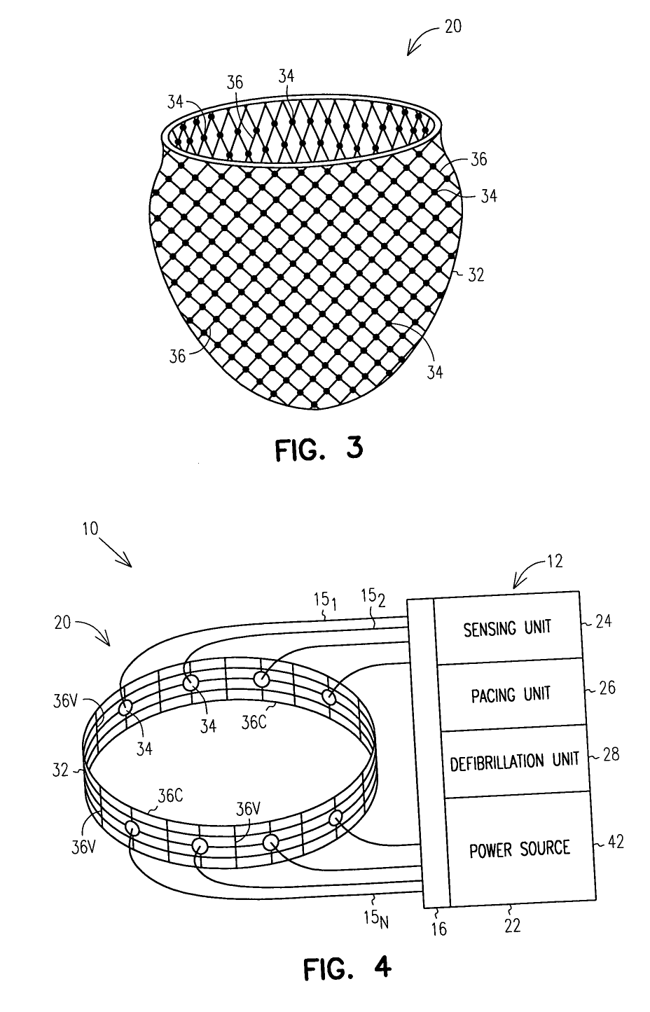

Electrode array 20 includes a support 32 on which are fixed a plurality of electrodes 34. The support 32 as shown in FIGS. 2 and 3 has a plurality of filaments 36 that cross to define a mesh structure having openings intermediate the filaments. Support 32 in this illustrated embodiment has a sack shape adapted to receive a therapeutic site in the interior of the sack. At the crossing points, the filaments 36 are fixed together. In an embodiment, the electrodes 34 fix the filaments together. In an embodiment, at least some of the filaments 36 are slidably connected together. The mesh structure generally conforms to the exterior shape of the location whereat it will be implanted. For example, the mesh structure shown in FIGS. 2 and 3 generally conforms to the shape of the lower three-quarters or less of the heart with an open top that will allow the electrode array to be mounted on the heart from the cardiac apex upwardly onto at least ventricles. In an embodiment, the height of the support is about one third the height of the heart. The support 32 is collapsible to aid in its insertion into the body by minimally invasive techniques. Each of the filaments 36 to which an electrode 34 is fixed includes an electrical conductor such as a wire to electrical communicate with circuits external to the electrode array 20. In an example, the filament 36 include a conductive wire center coated by an insulator, where the insulator is elastic. FIGS. 2 and 3 show that an electrode is positioned at each intersection of the filaments 36. It is within the scope of the present invention to eliminate electrodes from various filament intersections if it is known that an electrode is not required at that location for sensing or therapy. However as the electrodes 34 are independently addressable any individual electrode need not be activated.

The support 32 further provides a therapy function as a brace or restraint to the muscle to which it is attached. In an embodiment, the support 32 braces or restrains at least part of the epicardial surface of the heart. The support 32 defines an enclosure or sack having an interior in which the heart is received and an exterior. The filaments 36 further provide a passive restraint on the muscle so that it can not expand in a select direction more than a predetermined limit in an embodiment. When the muscle is a heart, the support can restrain expansion of the heart wall so that the heart chamber can not expand past its normal expansion and assist in efficient pumping. Thusly, the support 32 is used to assist in congestive heart failure treatments by preventing further development of the heart defects leading to congestive heart failure. The support 32 can be implanted into the pericardial space and onto the surface of the epicardium using minimally invasive techniques. The support 32 has flexible, elastic filaments 36 that will conform to the general shape of the heart. The filaments 36 provide a force on the order of ones of grams to hold the electrodes 34 in contact with the epicardial surface to ensure an electrical contact between the electrodes 34 and epicardial surface. Moreover, the elastic nature of the filaments should allow for the nature movement of the muscle, for example the heart. Accordingly, the filaments 36 allow the support 32 to expand and contract in a range of about 5% to about 12%. In an embodiment, the filaments 36 expand and contract about 10% or less. In an embodiment, the filaments 36 expand and contract about 15% or less.

FIG. 4 shows an embodiment of the system 10 having an integrated assembly of the CRM device 12 with the addressing circuit 16. CRM device 12 further includes a power source 42 such as a battery. The electrode array 20 is shaped as a cylindrical band that includes a plurality of circumferentially extending, elastic filaments 36C crossed by a plurality of vertically extending, elastic filaments 36V. The band is positioned around a portion of the muscle, e.g., heart. Electrodes 34 are positioned at at least some of the intersections of the vertical filaments 36V and the circumferential filaments 36C. In an embodiment, the electrodes 34 are positioned at every other vertical filament 36V. In an embodiment, the electrodes 34 are positioned only on the center circumferential filament 36C, essentially equidistance from the top and bottom of the cylindrical band. Filaments 36C and 36V remain on the outside of the electrode 34 so that the inwardly facing surface of the electrode is in direct contact with the muscle. In an embodiment, the electrode array encloses or surrounds the muscle and is in direct electrical contact to the muscle. In the illustrated FIG. 4 embodiment, there are eight electrodes 34. Each electrode 34 connected by a dedicated lead wire 15₁, 15₂, . . . 15_{N}. Lead wires 15₁, 15₂, . . . 15_{N} are connected to the addressing circuit 16. These electrodes 34 are located on the electrode array 20 so that they can be positioned at specific locations of the target tissue.

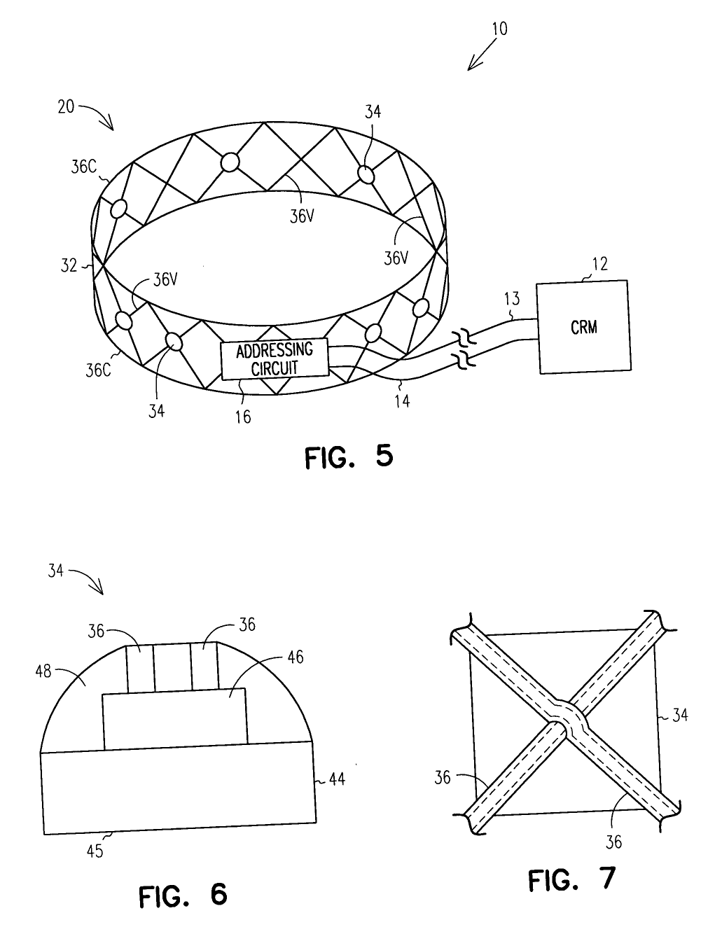

FIG. 5 shows an embodiment of system 10 having a band-shaped, generally cylindrical electrode array 20 with an addressing circuit 16 fixed on the array 20. Conductive leads 13, 14 connect addressing circuit 16 to the control device 12. In an embodiment, device 12 is only a power supply with the addressing, logic, memory and control circuits are in the circuit 16 fixed to the electrode array 20. In the FIG. 5 embodiment, the support 32 is formed from two circumferentially extending filaments 36C at the top and bottom and vertical extending filaments 36V extending between the filaments 36C. The vertical filaments 36V are not perpendicular to the circumferential filaments 36C and cross each other to provide intersections whereat the electrodes 34 are fixed. Electrical communication exists from the circuit 16 through the filaments 36V and 36C to electrodes 34 as required such that each electrode 34 is in communication with the circuit and individually selectable/actuatable.

FIG. 6 shows an electrode 34 according to an embodiment of the present invention. The electrode 34 includes a conductive plate 44 having a free surface 45 that is adapted to contact the tissue that will be controlled by the electrode. In an embodiment, plate 44 is made of a biologically inert, electrically conductive material. An electrode control circuit 46 is positioned on the other surface of the plate 44. A hermetic seal 48 encapsulates the electrode control circuit 46 to protect the surrounding tissue from the materials in the circuit 46 and protect the circuit from the environment.

Electrode control circuit 46 is electrically connected to an electrically conductive filaments 36. In the illustrated embodiment of FIG. 6 two filaments 36 are shown. One of these filaments is a column filament. The other filament is a row filament. Accordingly, the addressing circuit (not shown in FIG. 6) can activate this electrode by providing an on or “high” voltage level on both filaments 36. The electrode control circuit 46 can be a simple AND logic gate that produces a high output signal to electrode plate 44 when both filaments 36 are high. However, the electrode control circuit 46 is not limited to only an AND gate. Circuit 46 can include more advanced functions including anti-fuses, logic circuits, memory and other functions that can be defined by an application specific integrated circuit. Circuit 46, in an embodiment, includes a power source such as a capacitor that can be recharged by the conductive filaments. A processor composed of latches and transistors is powered by the power source. A memory is operably connected to the processor. The memory is adapted to store activation/deactivation codes. In operation, the controller 12 or addressing circuit 16 will send a serial activation code to the electrodes. The circuit 46 will latch the serial activation code and compare it to the stored activation code. If there is a match, then the electrode will activate. If the codes do not match then the electrode will remain inactive. A similar code system can be used to turn off an electrode.

The electrode memory in circuit 46 is adapted to store certain sensed signals in an embodiment. The tissue in contact with the electrode plate 44 produces an electrical signal that is transmitted by the plate to circuit 46. This sensed signal is stored in memory until it is transmitted from circuit 46 through conductive filaments 36 to external circuits 16 or 12.

In an embodiment, the circuit 46 may include thin film anti-fuses. Anti-fuses are not conductive until subjected to a breakdown voltage. When subject to the breakdown voltage the anti-fuse is conductive and the associated electrode is permanently active. Accordingly, certain electrodes are activated to sense and/or provide therapy by activating the anti-fuse.

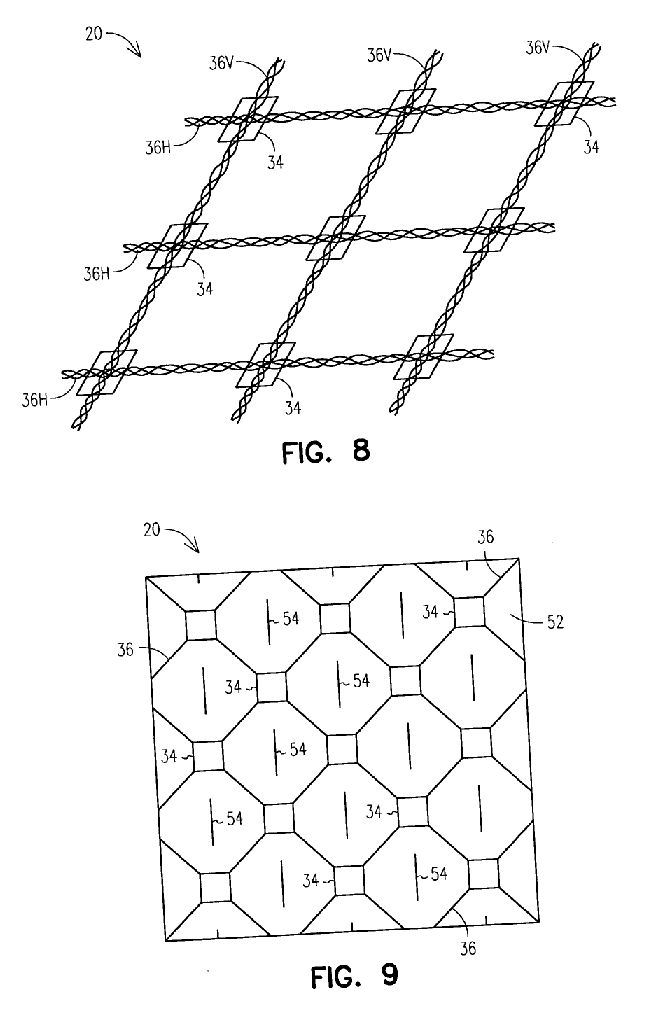

FIG. 7 shows a top view of an electrode 34 having two filaments 36 fixed thereto. The two filaments 36 cross on the upper surface of the electrode 34. The conductive wire in the filaments is shown in broken line. These conductive wires are in electrical communication with the electrode 34 but not in electrical communication with each other.

FIG. 8 shows a partial view of the electrode array 20 that includes a mesh formed by vertical filaments 36V and horizontal filaments 36H with openings between the filaments. Electrodes 34 are positioned at each intersection of filaments 36V, 36H. Each filament 36V, 36H is composed of a plurality of sub-filaments. In the illustrated embodiment of FIG. 8 there are three sub-filaments, however, it will be recognized that the invention is not limited to three sub-filaments. These sub-filaments are connected together. In an embodiment, the sub-filaments are woven together. In a further embodiment, the sub-filaments are fixed together, for example, by gluing, welding or other forms of joining. At least one of the vertical sub-filaments of each filament 36V includes a conductive core that is connected to electrodes 34 in the respective column in the electrode array 20. At least one of the horizontal sub-filaments of each filament 36H includes a conductive core that is connected to electrodes 34 in the respective row of the electrode array 20. These conductive sub-filaments are elastic in an embodiment. In a further embodiment, the non-conductive sub-filaments are elastic.

FIG. 9 shows a partial view of a further embodiment of the electrode array 20. A planar sheet 52 of an elastic material is provided with a plurality of conductive filaments 36 fixed thereon. The filaments include a first set of filaments with each filament of the first set being separate from each other and extending in a first direction. The filaments include a second set of filaments with each filament of the second set being separate from each other and extending in a second direction. The first direction is different than the second direction such that the filaments of the first set cross filaments of the second set whereat the electrodes 34 are positioned. Each electrode 34 is separately addressable as described herein. Intermediate the filaments 36 and the electrodes 34, the sheet 52 is cut, for example, at 54 as shown in FIG. 9. In an embodiment, the cuts form apertures in the sheet. Cuts 54 are linear in an embodiment. In a further embodiment, these cuts 54 extend parallel to the filaments of the first set. In an embodiment, the cuts 54 extend vertically between adjacent electrodes 34. Cuts 54 are in the diamond shaped in an embodiment. The cuts 54 allow the sheet 52 to expand and conform to the application site. In an embodiment, the application site is a muscle. In an embodiment, the application site is the heart.

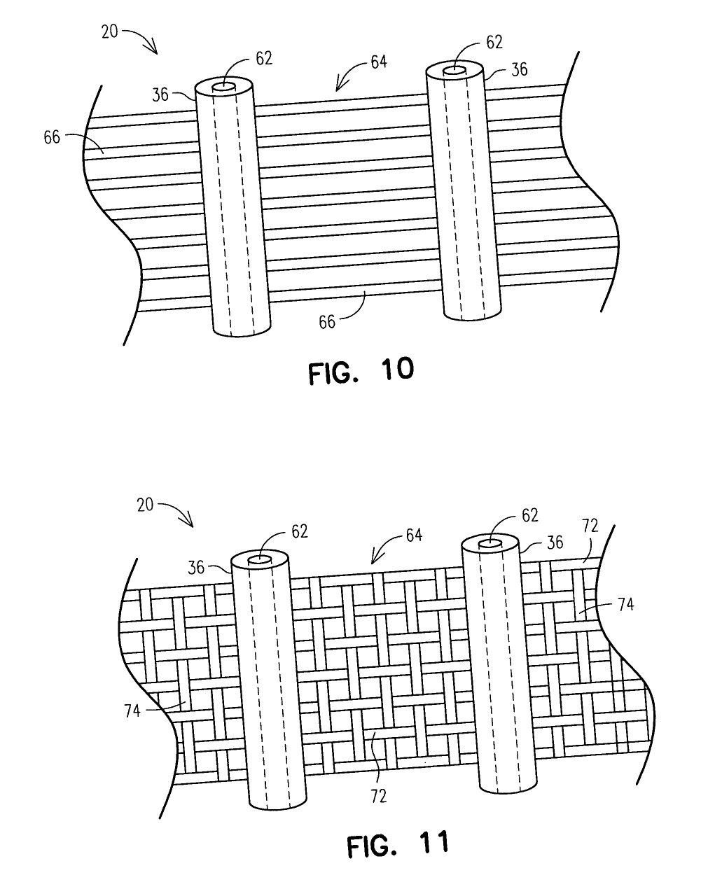

FIG. 10 shows a partial view of an electrode array 20 with vertical filaments 36 having a conductive core 62. The horizontal filaments are not shown for clarity of illustration. It is understood that the horizontal filaments will cross the vertical filaments 36 and electrodes will be fixed at the crossing point as described herein. A web 64 supports the filaments 36. The web 64 has horizontal strands 66 extending between the vertical filaments 36. Strands 66 are smaller than the filaments 36. Strands 66 are elastic to conform the electrode array 20 to the application site. This allows the filaments 36 to be essentially non-elastic but flexible.

FIG. 11 shows a partial view of an electrode array 20 with vertical filaments 36 having a conductive core 62. The horizontal filaments are not shown for clarity of illustration. Web 64 includes horizontal strands 72 and vertical strands 74 that are woven together. Web 64 is fixed to and extends between the filaments 36. Strands 72, 74 are smaller than the filaments 36. Strands 72, 74 are elastic to conform web 64 and, hence, the electrode array 20 to the application site. This allows the filaments to be essentially non-elastic but flexible. The density of the strands 72, 74 and the size of the opening between the strands is selected based on the elasticity of the strands and the desired elasticity of the electrode array 20.

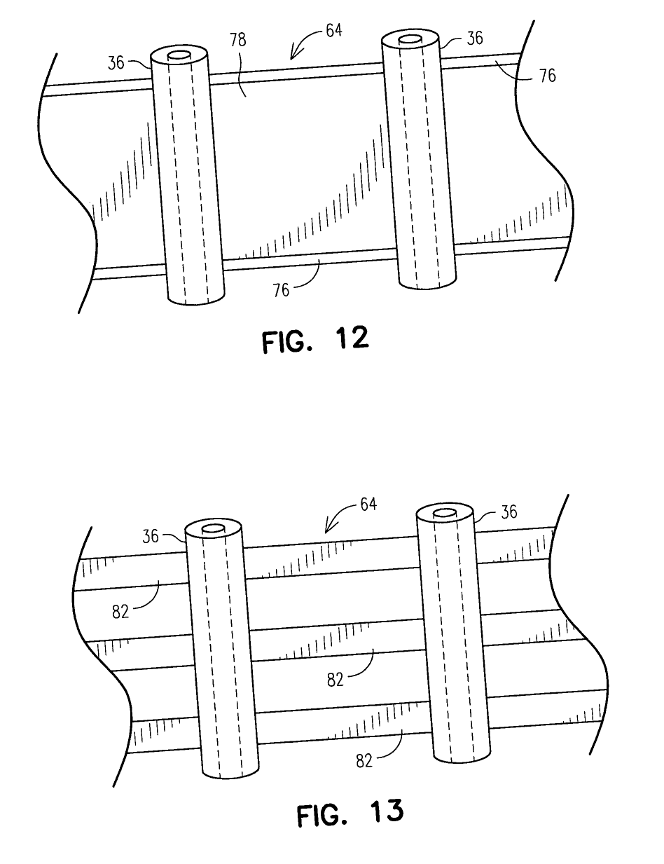

FIG. 12 shows filaments 36 on an elastic, continuous web 64 that consists of optional upper and lower strands 76 joined by a non-woven, solid sheet 78. Filaments 36 are fixed to the sheet 78.

FIG. 13 shows filaments 36 on an elastic web 64 formed from a series of separated, flat tapes 82. Tapes 82 may be non-woven.

Unlike conventional electrode implants that typically are only one or two electrodes, the present invention provides for greater than two electrodes. In an embodiment, the system 10 supports over ten electrodes, i.e., there are ten independently addressable electrodes 34. In an embodiment, the system 10 supports over one hundred electrodes. In some applications of the present invention the number of electrodes supported by the present system 10 may be expressed as 2^{N}, where N is a whole number greater than 1. This allows the present invention to adopt memory addressing for personal computer memory to the addressing of individual electrodes. Accordingly, in various embodiments, the electrodes 34 supported by the present system number one of 32, 64, 128, 256, 512, or 1028. A finer, and believed to be previously unattainable, resolution of sensing and therapy to a muscle such as the heart is attained. In an embodiment, the QRS waveform can be sensed as it travels across the heart tissue. This will allow a physician to more closely synchronize the therapy to the specific requirements of the patient. The electrode array 20 can provide a type of mono-polar pacing in which a single electrode 34 or a group of closely adjacent electrodes 34 provides the therapeutic signal. The electrode array 20 can provide bi-polar pacing in which any combination of two electrodes provides the therapeutic signal. Bi-polar pacing can further be provided by a first group of closely adjacent electrodes and a second group of closely adjacent electrodes with the first and second groups being separate from each other. In a further embodiment, the can 22 acts as the second electrode in a bi-polar pacing therapy. Any of the electrodes not providing a pacing signal can used for sensing or mapping the heart. Accordingly, the present system 10 provides an active medical device for sensing and/or providing a therapy.

When providing an electrical stimulation therapy over a period of time, scar tissue may develop on the electrodes. Scar tissue may block the stimulation/therapeutic signal emitted from the electrode 34. In a convention system having only one or two electrodes, it is necessary to replace or reposition the electrodes. This typically requires further surgery. Even with minimally invasive surgical procedures there are possible complications that may harm the patient. With the greater number of electrodes 34 in the present electrode array 20, when one electrode is ineffective in providing the therapeutic signal to the tissue, for example, due to scar tissue development, then a different, possibly directly adjacent electrode can be activated to provide the signal to the tissue. With the large numbers of electrodes 34 described herein adjacent electrodes are closely spaced and would provide the subject tissue with a substantially same stimulation/therapeutic signal as the blocked electrode. The present invention in various embodiments provides electrodes that are spaced millimeters or less from each other. The electrodes 34 in an embodiment are spaced from each other less than hundreds of micrometers from each other. The electrodes themselves may have a surface on the order of ones of millimeters by ones of millimeters. The adult human heart is generally the size of an adult fist. Accordingly, closely spacing the electrodes on the heart (epicardium) will provide a fine resolution for sensing and delivery of therapeutic signals.

In a further application of an embodiment of the present disclosure, the system 10 is used to sense the electrical activity of the heart or provide therapy related to remodeling. In a normal heart, the sinoatrial node, the heart's natural pacemaker, generates electrical impulses, called action potentials, that propagate through an electrical conduction system to various regions of the heart to excite the myocardial tissues of these regions. Coordinated delays in the propagations of the action potentials in a normal electrical conduction system cause the various portions of the heart to contract with synchrony to result in efficient pumping functions indicated by a normal hemodynamic performance. A blocked or otherwise abnormal electrical conduction and/or deteriorated myocardial tissue cause dysynchronous contraction of the heart, resulting in poor hemodynamic performance, including a diminished blood supply to internal organs. The condition where the heart fails to pump enough blood to meet the body's metabolic needs is known as heart failure.

The adult myocardium is incapable of repairing itself after an injury. Such an injury may result from, for example, myocardial infarction (MI), which is the necrosis of portions of the myocardial tissue resulted from cardiac ischemia. A condition in which the myocardium is deprived of adequate oxygen and metabolite removal due to an interruption in blood supply. The adult heart lacks a substantial population of precursor, stem cells, or regenerative cells. Therefore, after the injury, the heart lacks the ability to effectively regenerate cardiomyocytes to replace the injured cells of the myocardium. Each injured area eventually becomes a fibrous scar that is non-conductive and non-contractile. Consequently, the overall contractility of the myocardium is weakened, resulting in decreased cardiac output. As a physiological compensatory mechanism that acts to increase the cardiac output, the LV diastolic filling pressure increases as the pulmonary and venous blood volume increases. This increases the LV preload, including the stress on the LV wall before the LV contracts to eject blood. The increase of the LV preload leads to progressive change of the LV shape and size, a process referred to as remodeling. Remodeling is initiated in response to a redistribution of cardiac stress and strain caused by the impairment of contractile function in the injured tissue as well as in nearby and/or interspersed viable myocardial tissue with lessened contractility due to the infarct. The remodeling starts with expansion of the region of the injured tissue and progresses to a chronic, global expansion in the size and change in the shape of the entire LV. Although the process is initiated by the compensatory mechanism that increases cardiac output, the remodeling ultimately leads to further deterioration and dysfunction of the myocardium. Consequently, the myocardial injury, such as resulted from MI, results in impaired hemodynamic performance and a significantly increased risk of developing heart failure. The present system 10 provides electrical signals through the electrode array 20 to control remodeling.

While some embodiments described herein are directed to the diagnosis and/or therapy delivered to a heart, it will be understood that embodiments of the present invention may be adapted to the diagnosis or therapy delivery to other muscles. In an embodiment, the present system 10 is adapted to control the function of the bladder. The electrode array 20 is positioned around the detrusor to control its contraction and extension. Accordingly, the emptying of the bladder is controlled. It is foreseen that such a placement of the system 10 may replace surgical techniques such as some forms of conventional detrusorrhaphy. In a further embodiment, the present system 10 is adapted to control bowel function. The electrode array 20 is positioned around the anal sphincter to control its contraction and relaxation. Accordingly, the emptying of the bowel is controlled by selective activation of certain electrodes in the array 20. It is further foreseen that embodiments of the present system are adapted to control still further organs and muscles such as the brain, stomach, intestines, and skeletal muscles.

The filaments, support or conductors as described herein are manufactured from a material that generally does not generally adhere to the epicardium. In an embodiment, the filaments and the support are formed from polymers. In an embodiment, the polymers are solid, lubricious polymers. Examples of polymers include polyfluorocarbons and polyolefins. A specific example of the polymer is polytetrafluoroethylene (PTFE or TFE). Another example is ethylene-chlorofluoroethylene (ECTFE). Another example is fluorinated ethylene propylene (FEP). Another example is polychlorotrifluoroethylene (PCTFE). Another example is polyvinylfluoride (PVF). Still another example is polyvinylidenefluoride (PVDF). Still another example is polyethylene (LDPE, LLDPE, and HDPE). Yet another example is polypropylene. In an embodiment, the polymer is a nylon. In an embodiment, the polymer is a polysulphones.

The present invention may provide a drug therapy in addition to the electrical sensing, electrical therapy, and mechanical therapy described herein. A drug therapy is embedded in the mesh support 32 or in hermetic seal 48 of the electrodes 34. Examples of therapeutic agents include pharmacological agents and cellular material, which may be used in conjunction with each other. Examples of suitable pharmacological agents include, but are not limited to anti-arrhythmic drugs, thrombolytic agents, anti-inflammatory, anti-fibrotic agents, antibiotics, and steroids.

In this document, the terms “a” or “an” are used, as is common in patent documents, to include one or more than one. In this document, the term “or” is used to refer to a nonexclusive or, unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

It is to be understood that the above description is intended to be illustrative, and not restrictive. Although the use of the implantable devices has been in, for example, a cardiac stimulation system, the implantable device could as well be applied to other types of body stimulating systems. Many other embodiments will be apparent to those of skill in the art upon reviewing the above description. The scope should, therefore, be determined with reference to the appended claims, along with the full scope of equivalents to which such claims are entitled.

What is claimed is:

1. An implantable medical system, comprising:
a signal processor;
an addressing circuit operably connected to the signal generator; and
an electrode array operably connected to the addressing circuit, the electrode array including an elastic support, a plurality of electrical conductors secured to the elastic support, and a plurality of individually addressable electrodes connected to the support.

2. The system of claim 1, wherein the addressing circuit is adapted to activate selected ones of the plurality of electrodes.

3. The system of claim 2, wherein the signal processor provides a muscle stimulation signal transmitted though the addressing circuit and the electrical conductors of the support to the active ones of the plurality of electrodes.

4. The system of claim 3, wherein the electrode array is adapted to be placed around the heart such that the plurality of electrodes are in communication with the heart.

5. The system of claim 4, wherein the elastic support extends completely around a periphery of the heart.

6. The system of claim 5, wherein the elastic support has a sack shape adapted to receive at least a bottom portion of the heart in an interior of the elastic support.

7. The system of claim 4, wherein the signal processor produces a pacing signal at the active ones of the plurality of electrodes.

8. The system of claim 1, wherein the support includes a plurality of elastic strands.

9. The system of claim 8, wherein the plurality of elastic strands extend parallel to each other.

10. The system of claim 9, wherein the plurality of elastic strands are interwoven.

11. The system of claim 8, wherein the support includes a non-woven, elastic sheet.

12. The system of claim 11, wherein the sheet includes a plurality of apertures allowing the sheet to form the electrode array to a therapy site.

13. The system of claim 8, wherein the plurality of elastic strands are adapted to force the electrodes into contact with a muscle.

14. The system of claim 13, wherein the plurality of elastic strands exert a force of on an order of ones of grams.

15. The system of claim 1, wherein the support includes a releasable therapeutic agent.

16. The system of claim 15, wherein the therapeutic agent includes at least one selected from the group of anti-arrhythmic drugs, thrombolytic agents, anti-inflammatory, anti-fibrotic agents, antibiotics, and steroids.

17. An implantable electrode array, comprising:
a flexible support having a plurality of filaments, at least two of the filaments being electrically conductive; and
a plurality of electrodes fixed to the support and adapted to electrically communicate with the electrically conductive filament and with adjacent target tissue.

18. The electrode array of claim 17, wherein each of the filaments is electrically conductive.

19. The electrode array of claim 18, wherein the support is elastic to conform to the shape of the target tissue and hold the electrodes in contact with the target tissue.

20. The electrode array of claim 19, wherein the support is adapted to conform to the shape of the heart and hold the electrodes in contact with the epicardium.

21. The electrode array of claim 20, wherein the support is adapted to expand or contract 15% or less.

22. The electrode array of claim 21, wherein the filaments include a first group of filaments extending in a first direction and a second group of filaments extending in a second direction, the first group crossing the second group at intersections to form a mesh.

23. The electrode array of claim 22, wherein the electrodes are fixed to the intersections such that each electrode is individually addressable by the crossed first group filament and the second group filament.

24. The electrode array of claim 23, wherein each electrode includes a control circuit.

25. The electrode array of claim 24, wherein the control circuit includes logic to activate the electrode based on signals received from the filaments connected thereto.

26. The electrode array of claim 25, wherein the logic is an AND gate.

27. The electrode array of claim 24, wherein the control circuit include a memory.

28. The electrode array of claim 27, wherein the control circuit includes a processor in communication with the memory.

29. The electrode array of claim 21, wherein the support includes a web fixed to the filaments.

30. The electrode array of claim 21, wherein the plurality of electrodes number greater than or equal to 32.

31. The electrode array of claim 30, wherein the electrodes are spaced less than about one millimeter or less from each other.

32. The electrode array of claim 17, wherein the support includes a control circuit in communication with the conductive filaments, the control circuit being adapted to communicate with the electrodes thought the conductive filaments.

33. The electrode array of claim 32, wherein the control circuit provides activation signals to selectively activate electrodes.

34. A method for controlling implanted electrodes, comprising:
providing a support having flexible, elastic filaments around a target site;
sending an electrode addressing signal through the filaments to which the electrodes are attached; and
selectively activating electrodes based on the addressing signal.

35. The method of claim 34, wherein sending the electrode addressing signal includes receiving a serial signal at a control circuit on the support, demultiplexing the serial signal and sending the demultiplexed signal to the electrodes.

36. The method of claim 35, wherein selectively activating the electrodes includes providing a therapy signal to tissue adjacent the activated electrode.

37. The method of claim 36, wherein providing the therapy signal includes providing a cardiac rhythm management signal to heart tissue adjacent the active electrodes.

38. The method of claim 37, wherein selectively activating the electrodes includes sensing an event adjacent the electrode and transmitting a representative signal to a control circuit on the support and multiplexing the signal to transmit to a device remote the support through fewer communication lines than electrodes.
